(19) United States
(12) Patent Application Publication
(10) Pub. No.: US 2024/0180498 A1
(43) Pub. Date: Jun. 6, 2024

(54) TECHNIQUES FOR GENERATING ALERTS BASED ON A RELATIVE LOCATION OF A WEARABLE DEVICE

(71) Applicant: Oura Health Oy, Oulu (FI)

(72) Inventors: Petteri Lajunen, Oulu (FI); Mikko Pellervo Tuohimaa, Kempele (FI); Janne Lauri Käki, Helsinki (FI); Aino Elisa Sorvala, Oulu (FI)

(21) Appl. No.: 18/074,376

(22) Filed: Dec. 2, 2022

(51) Int. Cl. A61B 5/00; H04B 17/318; H04W 4/80

(52) U.S. Cl. CPC A61B 5/742; A61B 5/6802; A61B 5/6826; A61B 5/7415; H04B 17/318; H04W 4/80

(57) ABSTRACT

Methods, systems, and devices that support techniques for generating alerts based on a relative location are described. A method may include acquiring physiological data associated with a user via a wearable device, the physiological data including at least temperature data, acceleration data, and photolethysmogram (PPG) data, or a combination of data. The method may include determining that the user is not wearing the wearable device based on the physiological data. The method may include determining a signal strength associated with a wireless connection between the wearable device and a user device associated with the user based on the user not wearing the wearable device. The method may include causing a graphical user interface (GUI) of the user device to display notification associated with the wearable device based on the signal strength failing to satisfy a threshold signal strength.

### FIELD OF TECHNOLOGY

The following relates to wearable devices and data processing, including techniques for generating alerts based on a relative location of a wearable device.

### BACKGROUND

Some wearable devices may be configured to collect data from users associated with including temperature data, acceleration data, or photoplethysmography (PPG) data, and the like. However, a wearable device may become separated from the user, and the user may be unable to locate the wearable device.

### BRIEF DESCRIPTION OF THE DRAWINGS

FIG. 1 illustrates an example of a system that supports techniques for generating alerts based on a relative location of a wearable device in accordance with aspects of the present disclosure.

FIG. 2 illustrates an example of a system that supports techniques for generating alerts based on a relative location of a wearable device in accordance with aspects of the present disclosure.

FIG. 3 illustrates an example of a wear state diagram that supports techniques for generating alerts based on a relative location of a wearable device in accordance with aspects of the present disclosure.

FIG. 4 illustrates an example of a user diagram that supports techniques for generating alerts based on a relative location of a wearable device in accordance with aspects of the present disclosure.

FIG. 5 illustrates an example of a graphical user interface (GUI) diagram that supports techniques for generating alerts based on a relative location of a wearable device in accordance with aspects of the present disclosure.

FIG. 6 illustrates a block diagram of an apparatus that supports techniques for generating alerts based on a relative location of a wearable device in accordance with aspects of the present disclosure.

FIG. 7 illustrates a block diagram of a wearable device manager that supports techniques for generating alerts based on a relative location of a wearable device in accordance with aspects of the present disclosure.

FIG. 8 illustrates a diagram of a system including a device that supports techniques for generating alerts based on a relative location of a wearable device in accordance with aspects of the present disclosure.

FIGS. 9 through 11 illustrate flowcharts showing methods that support techniques for generating alerts based on a relative location of a wearable device in accordance with aspects of the present disclosure.

### DETAILED DESCRIPTION

Wearable devices may be used to collect, monitor, and track physiological data associated with a user based on sensor measurements performed by a wearable device (e.g., a wearable ring device, a watch, a necklace, or any other wearable device). Examples of physiological data may include temperature data, acceleration data, heart rate data, photoplethysmogram (PPG) data, bioimpedance data, electrocardiogram (ECG) data, bioimpedance data, and the like. The physiological data collected, monitored, and tracked via the wearable device may be used to gain health insights about the user, such as the user's sleeping patterns, activity patterns, and the like. However, in some cases, the wearable device may become separated from the user, such as if the user misplaces the wearable device. The user may be unable to locate the wearable device, and thus may incur cost from replacing the wearable device, may lose physiological data, or both.

Further, some systems may determine a separation between a user and a device exceeds a threshold and may provide information to a user to notify the user that the device is lost. However, in some cases, the wearable device may not actually be lost, and the notification may be unnecessary. For instance, a user device may determine that the distance between a wearable device and the user device exceeds a threshold distance and may trigger the notification, but the user may have intentionally left the user device at another location (e.g., the home of the user, the workplace of the user), and may still know the location of the device.

To prevent loss of a wearable device and false notifications that the wearable device is lost, the wearable devices may be configured to collect data to determine when a user may be separated from the wearable device. In some examples, the user may carry a user device (e.g., a phone, a computer, a tablet) that receives data, such as the physiological data, from the wearable device. In some cases, the user device may receive data from the wearable device that may provide for the user device to determine that the user has been separated from the wearable device. Additionally, or alternatively, the user device may provide data (e.g., motion data) that may indicate whether the wearable device is lost, such as by comparing movement data from each of the wearable device and the user device.

For example, the user device may determine a distance between the user and the wearable device has exceeded a threshold (e.g., based on a signal strength from the wearable device). Additionally, or alternatively, the user device may detect a loss in skin contact, such as by a change in sensor data from the wearable device that indicates ambient environment instead of user data. The user device may detect or otherwise determine that the user is not wearing the wearable device based on the data, and may subsequently alert the user if a signal strength of a wireless connection between the wearable device and the user device fails to satisfy a threshold signal strength.

In some examples, the user device may alert the user of the separation from the wearable device, and the user may enable a mode (e.g., a lost mode, a find mode, a stolen mode) at the user device to locate the wearable device. A graphical user interface (GUI) of the user device may display a location of the wearable device to the user. In some examples, the location may be a current location or a last known location of the wearable device (e.g., location of the user device when the user device was last communicatively coupled with the wearable device). Additionally, or alternatively, the user device may automatically trigger an alert (e.g., a warning, a notification) when the user exceeds a threshold distance between the user and the wearable device and the data from the wearable device indicates that the user is not wearing the wearable device.

Aspects of the disclosure are initially described in the context of systems supporting physiological data collection from users via wearable devices. Aspects of the disclosure are then described in the context of wear state diagrams, user diagrams, and GUI diagrams. Aspects of the disclosure are further illustrated by and described with reference to apparatus diagrams, system diagrams, and flowcharts that relate to techniques for generating alerts based on a relative location.

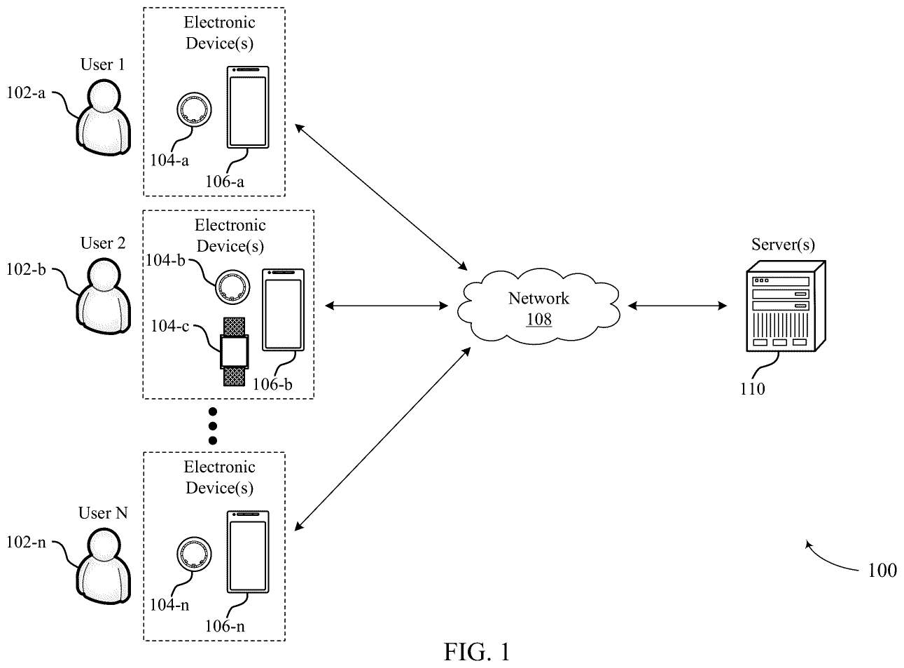

FIG. 1 illustrates an example of a system 100 that supports techniques for generating alerts based on a relative location of a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible, or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IOT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as PPG waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled with one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled with the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108 and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state: 2) circadian rhythms: 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used): 5) multi-day ovarian rhythms in women and spermatogenesis rhythms in men: 6) lunar rhythms (relevant for individuals living with low or no artificial lights): and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to capture the dynamic physiological baselines of an individual more accurately or group of individuals.

In some aspects, the respective devices of the system 100 may support techniques that leverage physiological data, such as temperature data, acceleration data, and PPG data, measured at a wearable device 104, and light capabilities, auditory capabilities, a GUI, or any combination thereof, of a user device 106 to support generating alerts based on a relative location of the wearable device 104. For example, a user device 106 may use the physiological data from the wearable device 104 to determine an “on” or an “off” wear state (e.g., a wearable device 104 on a user 102, a wearable device 104 off of the user 102) of the wearable device 104. For the purposes of the present disclosure, the term “on wear state” may refer to the state of a wearable device 104 when the wearable device 104 is being worn by the user. Comparatively, the term “off wear state” may refer to the state of the wearable device 104 when the wearable device 104 is not being worn by the user.

For instance, a user device 106 may compare one or more physiological data measurements from a wearable device 104 to one or more threshold values (e.g., a threshold temperature, a threshold acceleration, a threshold PPG measurement that may indicate that the user 102 is not wearing the wearable device 104) to determine the wear state of the wearable device 104.

In some examples, the wear state of the wearable device 104 may depend on the temperature data, such that the wearable device 104 is in an “on” wear state when the temperature data is within a threshold range relative to a temperature of the user 102. Comparatively, the wearable device 104 may be in an “off” wear state when the temperature data is outside of the threshold range (e.g., a temperature of the ambient environment). Similarly, the user 102 may be walking and may drop the wearable device 104 from a height above the ground. The accelerometer data may detect an abnormal acceleration of the wearable device 104 (e.g., an acceleration that exceeds a threshold value) because of gravity, which may indicate to the user device 106 that the wearable device 104 is in an off wear state. Additionally, or alternatively, the PPG data from one or more light sources (e.g., LEDs) and photodetectors may collect measurements of blood pressure, oxygen levels, and heart rate that are abnormally low or high for a user 102 (e.g., below a threshold value relative to readings for a user 102), indicating to the user device 106 that the wearable device 104 is in an off wear state off.

In some aspects, the user device 106 may determine a signal strength of a wireless connection (e.g., a Bluetooth connection) between the wearable device 104 and the user device 106 when the user device 106 determines the user 102 is not wearing the wearable device 104 from the data (e.g., when the wearable device 104 is in an off wear state). In some cases, the user device 106 may compare the signal strength to a threshold signal strength value to determine a distance between the user device 106 and the wearable device 104, which may also be referred to as a relative location of the wearable device 104. In some implementations, a GUI of the user device 106 may display an alert to the user 102 associated with the wear state of the wearable device 104, such as when the signal strength fails to meet the threshold signal strength (e.g., alert a user 102 when the wearable device 104 is 5 meters away from the user device 106, 10 meters away from the user device 106, or the like). In some cases, the alert at the user device 106 may include information of current and last known locations of the wearable device 104, such as stored location data indicating a geographical location of the wearable device 104 (e.g., relative to the user device 106).

Additionally, or alternatively, the wearable device 104, the user device 106, or both may utilize sound or visible light components to alert the user 102 of the relative location of the wearable device 104. In some examples, the user device may configure one or more parameters of the sound or the visible light (e.g., a volume of the sound, a frequency of the sound, a pattern of the sound or visible light, an intensity of the visible light, a wavelength or color of the visible light, a periodicity of a blinking pattern, or the like) based on the distance between the user device 106 and the wearable device 104, a power level of the wearable device 104, or both.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

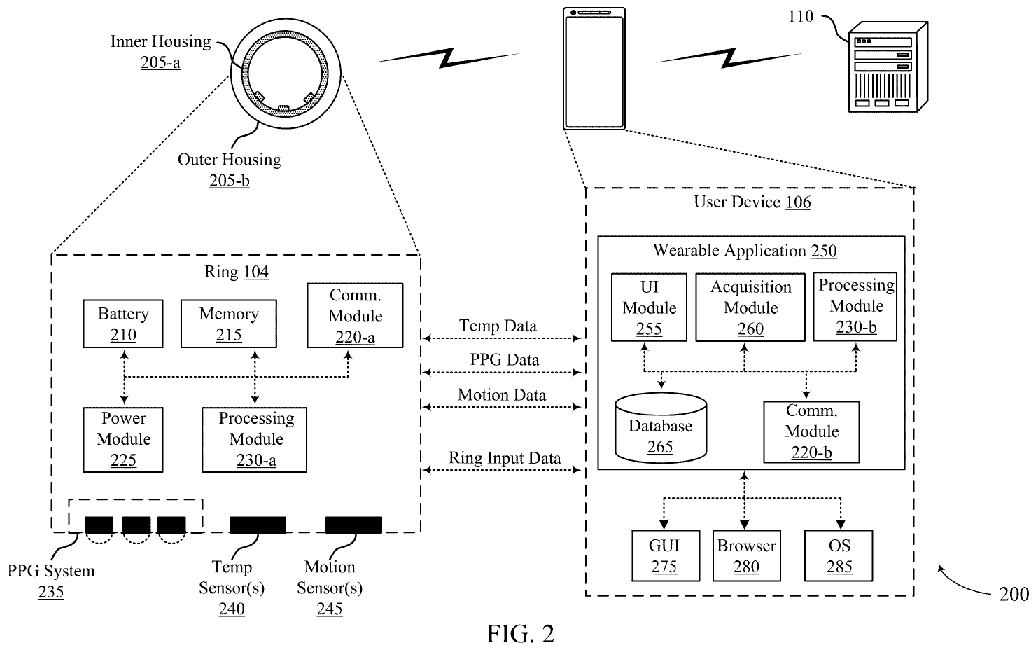

FIG. 2 illustrates an example of a system 200 that supports techniques for generating alerts based on a relative location of a wearable device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or another sensor) may be fabricated. In another specific example, a temperature sensor 240 (or other sensor) may be attached to a user's finger (e.g., using a clamp, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104 and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercises (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230)-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1-minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five-minute time period for storage, and then subsequently erase the one-minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, or alternatively, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed and may be calculated using the efficiency average of long sleep periods (e.g., primary sleep period) of the sleep day, weighted by a duration of each sleep period. The “restfulness” contributor may indicate how restful the user's sleep is and may be calculated using the average of all sleep periods of the sleep day, weighted by a duration of each period. The restfulness contributor may be based on a “wake up count” (e.g., sum of all the wakeups (when user wakes up) detected during different sleep periods), excessive movement, and a “got up count” (e.g., sum of all the got ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day and may be calculated using the average of all sleep periods of the sleep day, weighted by a duration of each period.

By way of another example, a user's overall Readiness Score may be calculated based on a set of contributors, including sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the respective devices of the system 200 may support techniques that leverage physiological data, such as temperature data, acceleration data, and PPG data, collected via a wearable device 104, and light capabilities, auditory capabilities, a GUI, or any combination thereof, of a user device 106 and/or the wearable device 104 to support generating alerts based on a relative location of the wearable device 104.

For example, a user device 106 may utilize data collected by a wearable device 104, such as a ring, watch, necklace, or any other wearable device 104, to determine if a user 102 is wearing the wearable device 104 (e.g., determine the wear state of the wearable device 104). In some aspects, the user device 106 may acquire data from the wearable device 104 to determine that the user 102 is not wearing the wearable device 104 (e.g., wearable device 104 is in an off wear state). In such examples, a user device 106 may determine a signal strength of a wireless connection between the wearable device 104 and the user device 106.

By determining the signal strength when the wearable device 104 is in an off wear state, the user device 106 may generate alerts associated with a relative location of the wearable device 104. Moreover, by triggering alerts based on the wear state of the wearable device 104, techniques described herein may reduce a frequency that “false positive” alerts are provided to users (e.g., prevent an alert from being generated when a user goes for a run wearing the wearable device 104, but leaves their phone at home) In some cases, the user device 106 may indicate for the wearable device 104 to emit a visible light, or the user device 106 may emit a sound from one or more components. Additionally, or alternatively, the user device 106 may display one or more notifications to a user 102 via a GUI of the user device 106.

For example, the system 200 may support a user device generating alerts that notify the user 102 of a relative location of the wearable device 104 based on physiological data that includes temperature data from one or more temperature sensors 240, motion data from one or more motion sensors 245, and PPG data from a PPG system 235, or any other sensor data from the wearable device 104. In some aspects, the user device 106 may use temperature data from the one or more temperature sensors 240 to determine if the user 102 is wearing the wearable device 104 (e.g., the wear state of the wearable device 104). In some examples, the temperature data from the temperature sensor 240 may include a temperature of the user 102 when the wearable device 104 is worn by the user 102 and a temperature of the ambient environment when the wearable device 104 is not worn by the user 102. In some cases, the user device 106 may use the temperature data to determine a wear state of the wearable device 104, such that the wearable device 104 is in an on wear state may when the temperature data is within a temperature rang associated with the user 102, and in an off wear state when the temperature data is within a temperature range associated with the ambient environment (or otherwise outside of the temperature range associated with the user).

In some cases, the user device 106 may use the motion data, such as acceleration data, from the one or more motion sensors 245 to determine if the user 102 is wearing the wearable device 104. For example, the acceleration data may include motion data of the user 102, such as a body part of the user 102 the wearable device 104 is on. That is, if the wearable device 104 is a ring on a human finger, the motion sensors 245 may detect an arm swinging while a user 102 is walking or running, motion during a sleep duration of the user 102, or any other motion of the user 102, which may be relatively nominal changes in movement. However, the acceleration data may include changes in motion data when the user 102 is separated from the wearable device 104. For example, the user 102 may drop the wearable device 104 from a height above the ground, causing the acceleration data to reflect an anomaly (e.g., relatively large acceleration value). In other words, the acceleration data as the wearable device 104 drops from a height above the ground may satisfy a threshold acceleration value (e.g., exceeding the threshold acceleration, or otherwise identifiable as a free fall acceleration), where the threshold acceleration value is greater than a maximum acceleration value for a user 102. In such cases, the system 200 may recognize the acceleration data as indicating the user has dropped the wearable device 104, and that the wearable device 104 is now likely in an off wear state.

In some cases, the user device 106 may use the PPG data from the PPG system 235 to determine if the user 102 is wearing the wearable device 104. In some examples, the PPG system 235 may use one or more light sources (e.g., LEDs) and photodetectors near the surface of skin to measure the volumetric variations of blood flow of the user 102. In some cases, the wearable device 104 may send one or more PPG measurements to a user device 106, and the user device may determine the PPG measurements indicate that the user 102 is not wearing the wearable device 104 (e.g., wearable device 104 is in an off wear state). For example, the PPG measurements may be below a threshold value for a human. For instance, the PPG data may include heart rate and/or SpO2 measurements that reflect more anomaly readings, such as relatively low heart rate and/or SpO2 when compared with minimum heart rate and/or SpO2 measurements of the user 102. In some aspects, if the PPG data is below the threshold value, the system 200 may determine that the wearable device 104 may be in an off wear state.

Additionally, or alternatively, the user device 106 may determine the wearable device 104 is in an off state based on ECG data, bioimpedance data, or any other data from the wearable device 104.

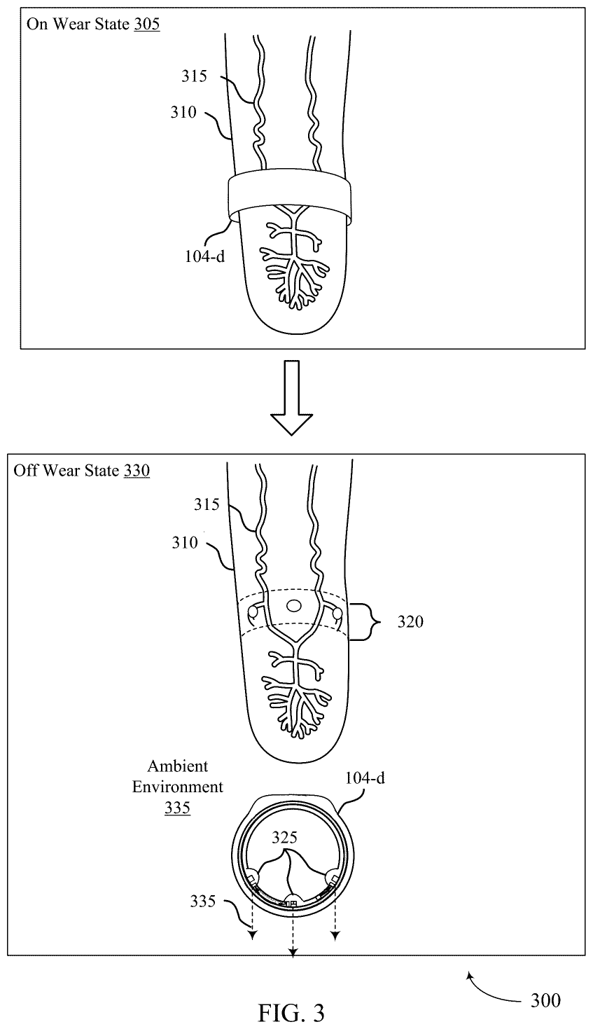

FIG. 3 illustrates an example of a wear state diagram 300 that supports techniques for generating alerts based on a relative location of a wearable device in accordance with aspects of the present disclosure. In some examples, the wear state diagram 300 may implement, or be implemented by, aspects of the system 100 and the system 200, and may include a wearable device 104-d, which may be an example of a wearable device 104 as described with reference to FIGS. 1 and 2. The wear state diagram 300 may illustrate an on wear state 305 and an off wear state 330 of the wearable device 104-d.

Although the wearable device 104-d is illustrated as a ring in FIG. 3, the wearable device 104-d may be any example of a wearable device (e.g., a watch, a necklace, an earbud, or the like). In some cases, one or more users (e.g., one or more users 102 as described with reference to FIGS. 1 and 2) may use different wearable devices with multiple shapes, sizes, colors, and properties. In some cases, the wearable device 104-d may couple to a human body component 310 for performing physiological data measurements via one or more wearable device sensors 325, such as at the coupling site 320. That is, the wearable device sensors 325 may contact skin of a user at the coupling site 320 to perform measurements of fluid flow through one or more veins 315 of the user.

The wearable device sensors 325, which may be referred to as sensors, may be located on the inside of the wearable device 104-d. In some cases, there may be any quantity of wearable device sensors 325 in locations along the wearable device 104-d. The sensors may vary in size and distance from each other. In some examples, the wearable device sensors 325 may include a PPG system (e.g., LEDs and photodetectors), thermal sensors, movement sensors, or the like, for detecting optical properties, thermal properties, and movement of a user, as described with reference to FIG. 2. For example, the human body component 310 may have fluid flow (e.g., blood flow) through one or more veins 315. The wearable device 104-d may collect measurements relating to the fluid flow or additional measurements (e.g., heart rate, blood pressure, or the like) from the veins 315, such as by using internal stray light measurements within the human body component 310 or other properties of the human body component 310. As described with reference to FIG. 2, in some examples, the LED sensors may measure PPG signal data, such as pulse waveforms and data calculated based on the pulse waveforms (e.g., heart rate values, body impedance, ECG data, IBI values, HRV values, and respiratory rate values). In some other examples, the LED sensors may measure blood oxygen levels (e.g., SpO2).

In some cases, the wearable device 104-d may report one or more measurements (e.g., physiological data) from the wearable device sensors 325 to a user device. The user device may determine a wear state of the wearable device 104-d from the one or more measurements. For example, the user device may determine that the user is wearing the wearable device 104-d, which may be an on wear state 305. Similarly, the user device may determine that the user is not wearing the wearable device 104-d, which may be an off wear state 330. In some cases, such as in the on wear state 305, the PPG signal data, temperature data, motion data, or any other data from the wearable device sensors 325 may include values within a threshold range for the user (e.g., typical measurements of the user). In some other cases, such as in the off wear state 330, the PPG signal data, temperature data, motion data, or any other data from the wearable device sensors 325 may include values outside of the threshold range for the user (e.g., atypical measurements of the user). Thus, the user device may use the values of the PPG signal data, temperature data, motion data, or any other data from the wearable device sensors 325 to determine whether the wearable device 104-d is in the on wear state 305 or the off wear state 330, as described with reference to FIG. 2.

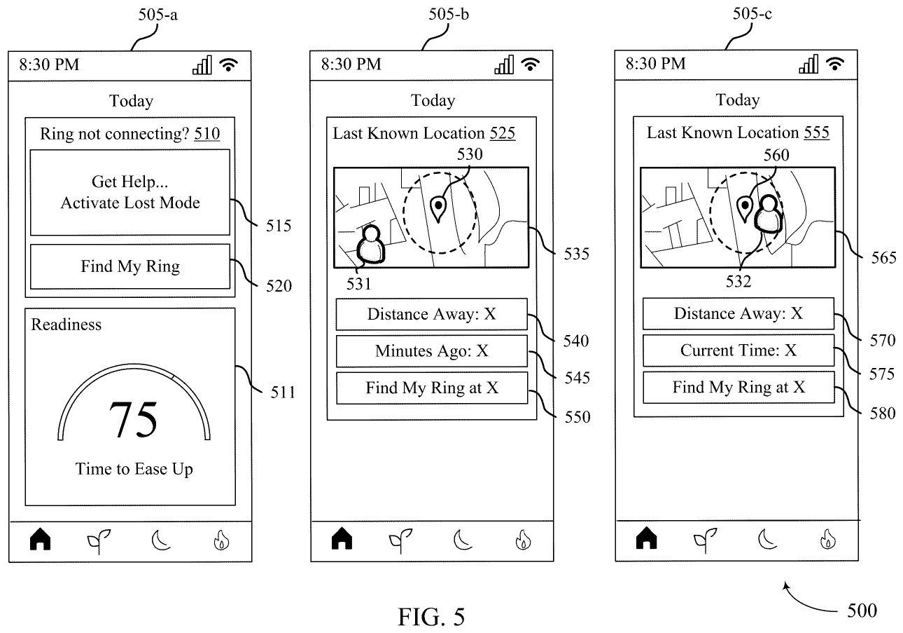

For example, the user device may use motion data or acceleration data from the wearable device sensors 325 (e.g., motion sensors) to determine if the user is wearing the wearable device 104-d. In the on wear state 305, the acceleration data may include changes in movement within a threshold (e.g., a maximum range of movement or acceleration) for a user. In some examples, such as in the wear state diagram 300, the user may drop the wearable device 104-d from a height above the ground, which may cause the data to exceed the threshold movement for the user. That is, the acceleration data as the wearable device 104-d drops from the high above ground may satisfy a threshold acceleration value, where the acceleration value is greater than a maximum acceleration value for the user. In such instances, the maximum acceleration value has been exceeded, which may indicate to the user device that the wearable device 104-d is in an off wear state 330. Similarly, if the wearable device sensors 325 fail to detect movement of a user for a defined duration, the user device may determine that the wearable device 104-d is in the off wear state 330. The system may generate an alert notifying the user that the wearable device 104-d is separated from the user, may display a relative location of the wearable device 104-d, or both, which is described in further detail with respect to FIG. 5.

Additionally, or alternatively, the user device 106 may use temperature data from the wearable device sensors 325 (e.g., temperature sensors) to determine if the user is wearing the wearable device 104-d. For example, when in the on wear state 305, the temperature sensors may measure the user's skin temperature of the human body component 310. The skin temperature may fluctuate according to threshold variations for the user. A temperature of the ambient environment 335 may be outside of the threshold variations for the user (e.g., a maximum and minimum temperature for the user). Thus, the user device may detect an off wear state 330 of the wearable device 104-d when the temperature data from the wearable device 104-d is outside of (e.g., exceeds or fails to satisfy) the threshold variations for the user, such as when the temperature data is of the ambient environment 335. When the user device detects the off wear state 330 of the wearable device 104-d, the system may generate an alert notifying the user that the wearable device 104-d is separated from the user, may display a relative location of the wearable device 104-d, or both as described with further detail with respect to FIG. 5.

Additionally, or alternatively, the user device may use PPG data from the wearable device sensors 325 (e.g., the PPG system) to determine if the user is wearing the wearable device 104-d. For example, when in the on wear state 305, the PPG system may report data associated with the fluid flow (e.g., heart rate data, SpO2 data, or the like) of the veins 315 in the human body component 310. The PPG data about the fluid flow through the veins 315 may fluctuate according to threshold variations for the user, such as based on an activity level of the user (e.g., when the user is working out, when the user is sleeping, etc.). In some examples, the user device may determine that the wearable device 104-d is in an off wear state 330 based on one or more PPG measurements being outside of the threshold variations, such as PPG measurements below a minimum value for a user (e.g., relatively low heart rate and/or SpO2 when compared with minimum heart rate and/or SpO2 measurements of the user). Additionally, or alternatively, a PPG pulse waveform, pulse variation, pulse quality metrics, or other characteristics may be outside of the threshold variations, indicating the off wear state 330. When the user device 106 detects the off wear state 330 of the wearable device 104-d, the system may generate an alert notifying the user that the wearable device 104-d is separated from the user, may display a relative location of the wearable device 104-d, or both as described with further detail with respect to FIG. 5.

In some examples, the user may intentionally remove the wearable device 104-d from the human body component 310. For example, the user may remove the wearable device 104-d when for activities where the wearable device 104-d may become damaged, to place on a charger, or the like. In such examples, the user device 106 may determine that the wearable device 104-d is at a specified place (e.g., located on the charger), such as based on location data from the wearable device 104-d, inherent charging state information of the wearable device 104-d, or the like, and may not trigger a generated alert to notify the user of the relative location of the wearable device 104-d. In some examples, the system may determine that the wearable device 104-d is in a known location (on the charger, on the user's nightstand, etc.) even when the user is separated from the device and may not trigger the location alert to the user.

In some other examples, the user may become separated from the wearable device 104-d unintentionally. For example, the user may become separated from the wearable device 104-d due to dropping the wearable device 104-d, taking the wearable device 104-d off for an unintentional duration (e.g., for too long), or the like. Additionally, or alternatively, the user may be unaware of the location of the wearable device 104-d and may benefit from an alert that notifies the user of the relative location of the wearable device 104-d.

In some implementations, a system may use the wear state of the wearable device 104-d to generate alerts for a relative location of the wearable device 104-d. For example, the system may not trigger notifications to the user about the location of the wearable device 104-d if the wearable device 104-d is in an on wear state 305 (e.g., when the user is wearing the wearable device 104-d). For instance, the user may be wearing the wearable device 104-d (e.g., on wear state 305) as the user goes for a run and may leave their user device 106 at home during the run. In this example, even though the user device 106 may determine that the wearable device 104 is traveling further and further from the wearable device, the system may nonetheless refrain from generating an alert based on determining that the wearable device 104-d is in the on wear state 305.

In some other examples, the system may trigger notifications to the user about the location of the wearable device 104-d if the wearable device 104-d is in an off wear state 330 (e.g., when the wearable device 104-d is separated from the user). In some examples, the system may determine the wear state of the wearable device 104-d by utilizing measurements from the wearable device 104-d. For example, the user device 106 may determine the wearable device 104-d is in the off wear state 330 by using the wearable device sensors 325, such as by comparing the measurements from the wearable device sensors 325 to one or more thresholds values (e.g., one or more baselines, one or more reference values) that are based on physiological data of the user, as described with reference to FIG. 2.

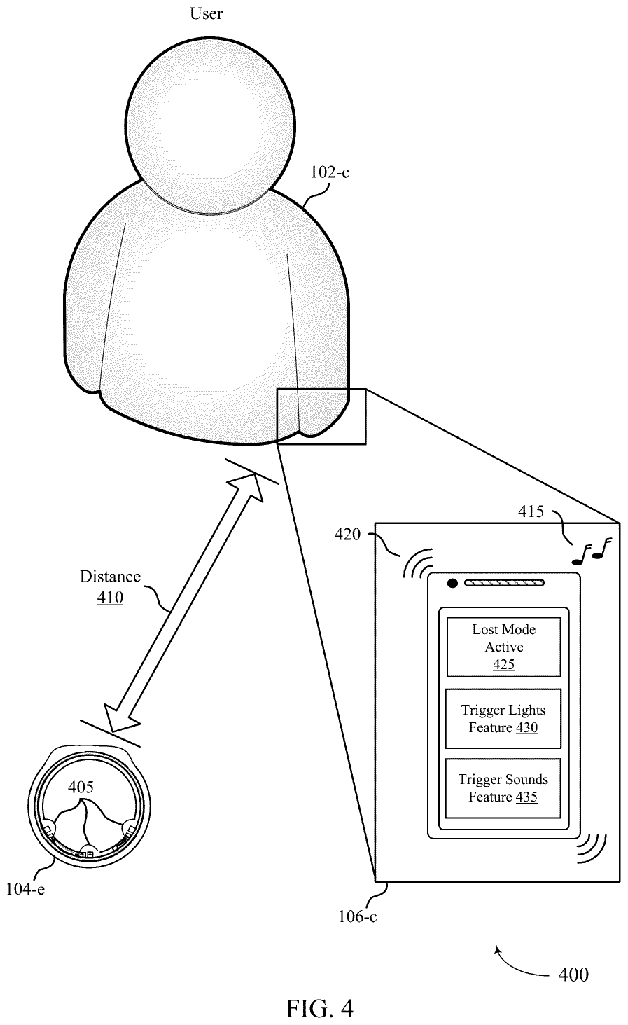

FIG. 4 illustrates an example of a user diagram 400 that supports techniques for generating alerts based on a relative location of a wearable device in accordance with aspects of the present disclosure. The user diagram 400 may implement, or be implemented by, aspects of the system 100, the system 200, the wear state diagram 300, or any combination thereof. For example, the user diagram 400 may include a user 102-c, a wearable device 104-e, and a user device 106-c, which may be examples of the corresponding devices as described with reference to FIGS. 1-3.

The user diagram 400 may illustrate an example of a user device 106-c determining that a user 102-c is not wearing a wearable device 104-e (e.g., the wearable device 104-e is in an off wear state) based on physiological data acquired from one or more sensors 405 of the wearable device 104-e, which may be examples of the sensors 325 as described with reference to FIG. 3 (e.g., temperature sensors, ECG sensors, bioimpedance sensors, motion sensors, and/or a PPG system).

In some examples, the user 102-c may input a threshold distance 410 between the user 102-c and the wearable device 104-e for triggering one or more alerts for a relative distance between the wearable device 104-e and the user device 106-c. In some cases, the threshold distance 410 may be indicated by the user 102-c based on a range of the Bluetooth connectivity between the user device 106-c and the wearable device 104-e. In some other examples, the threshold distance 410 to trigger the alerts may be otherwise defined, such as set (e.g., by the user 102-c) to a range of 10 meters, 5 meters, or the like based on the transmission capability of the wearable device 104-e. In some examples, the threshold distance 410 may be directly related to a strength of the wireless connection 420 between the wearable device 104-e and the user 102-c.

In some aspects, the user device 106-c may determine the wearable device 104-e is in an off wear state, as described with reference to FIG. 3. The user device 106-c may determine a signal strength for a wireless connection 420 between the wearable device 104-e and the user 102-c, which may be directly related to the threshold distance 410. For example, in some cases, upon determining that the wearable device 104-e is in the off wear state, the user device 106-c may evaluate the signal strength of the connection in order to determine whether or not the user device 106-c should generate an alert for the user.

For instance, the signal strength may indicate a lower signal strength when the distance between the user 102-c (e.g., user device 106-c) and the wearable device 104-c increases. In some other examples, the signal strength between the user 102-c (e.g., user device 106-c) and the wearable device 104-e may indicate a higher signal strength when the distance decreases. In some implementations, the signal strength may be directly proportional to one or more transmit capabilities for a wireless connection 420 between the user device 106-c and the wearable device 104-e.

In some implementations, the user device 106-c may activate one or more modes to generate alerts for the relative location of the wearable device 104-e if the wearable device 104-e is within the threshold distance 410, is outside of the threshold distance 410, or both. For example, the signal strength for the wireless connection 420 may fail to meet the threshold signal strength if a distance between the wearable device 104-e and the user device 106-c exceeds the threshold distance 410. In some aspects, the user device 106-c may determine that the signal strength fails to meet the threshold signal strength, and the user device 106-c may notify the user 102-c of one or more options, or modes, via a GUI of the user device 106-c.

In some aspects, the user device 106-c may determine that the signal strength fails to meet the threshold signal strength (e.g., the wearable device 104-e is far from the user device 106-c) and may activate a lost mode 425 at the user device 106-c, which may also be referred to as a find mode or any other mode for the user device 106-c to find the wearable device 104-e. In some examples, the user device 106-c may receive an input from the user 102-c that selects one or more options via the GUI. For example, the GUI may include one or more geographical location services that the user 102-c may use to locate the wearable device 104-e. In some cases, the user 102-c may select a lost mode 425 option to activate the lost mode 425, and the user device 106-c may display one or more options for the user 102-c to use to attempt to locate the wearable device 104-e, which is described in further detail with respect to FIG. 5.

In some examples, the wearable device 104-e may be equipped with one or more LEDs, where the LEDs may operate using different wavelengths (e.g., red, blue, green, yellow; or the like). In some examples, one of the options that the user 102-c may select via the GUI of the user device 106-c is a trigger lights feature 430, which may activate one or more visible lights at the wearable device 104-e. In some examples, the wearable device 104-e may use one or more components (e.g., sensors, modules, LEDs) to emit a visible light when the trigger lights feature 430 is activated by a user 102-c. In some examples, one or more parameters of the trigger lights feature 430 may adjust the visible light based on the distance between the user 102-c and the wearable device 104-e, the power level of the wearable device 104-e, or both. For example, the user device 106-c may indicate to the wearable device 104-e to activate one or more visible light components if the power level of the wearable device 104-e satisfies a threshold (e.g., is relatively high). If the power level of the wearable device 104-e fails to satisfy the threshold (e.g., is relatively low), the GUI of the user device 106-c may remove the trigger lights feature 430 from a selection window for the user 102-c, may notify the user 102-c of the low battery level of the wearable device 104-e, or both.

In some aspects, the one or more parameters of the visible light may include an intensity of the visible light, a wavelength of the visible light, a blinking pattern of the visible light, a periodicity of the blinking pattern, or any combination thereof. In some examples, the user 102-c may use the trigger lights feature 430 to locate the wearable device 104-e in a relatively dark room (e.g., the wearable device 104-e may emit a red or green pattern to help the user 102-c find the wearable device 104-e in the dark). The user 102-c may scan the room to detect the location of the wearable device 104-e, such as depending on varying speeds of flashing visible lights or varying brightness of visible lights emitting from the wearable device 104-e. In some cases, the user 102-c may use the trigger lights feature 430 to locate the relative location of the wearable device 104-e.

In some other examples, the user 102-c may select a trigger sound feature 435 at the wearable device 104-e or the user device 106-c to activate a sound component to emit a sound 415. In some examples, the user 102-c may activate the trigger sounds feature 435 via a selection on the GUI at the user device 106-c to cause one or more components (e.g., sensors, modules) of the wearable device 104-e, the user device 106-c, or both, to emit the sound 415. In some examples, one or more parameters of the trigger sounds feature 435 may adjust the sound 415 based on the distance between the user device 106-c and the wearable device 104-e, a power level of the wearable device 104-e, or both. For example, the user device 106-c may indicate to the wearable device 104-e to activate one or more sound components to emit the sound 415 if the power level of the wearable device 104-e satisfies a threshold power level for the wearable device 104-e (e.g., is relatively high), or the user device 106-c may activate one or more sound components to emit the sound 415 if the power level of the user device 106-c satisfies a threshold power level for the user device 106-c (e.g., is relatively high). If the power level of the wearable device 104-e fails to satisfy the threshold power level for the wearable device 104-e (e.g., is relatively low), the user device 106-c may remove the trigger sounds feature 435 from a selection window for the user 102-c, may notify the user 102-c of the low battery level of the wearable device 104-e, or both. Similarly, if the power level of the user device 106-c fails to satisfy the threshold power level for the user device 106-c (e.g., is relatively low), the user device 106-c may remove the trigger sounds feature 435 from a selection window for the user 102-c, may notify the user 102-c of the low battery level of the user device 106-c, or both.

In some aspects, the one or more parameters of the sound 415 may include a volume of the sound 415, a frequency of the sound 415, a pattern of the sound 415, or a combination of parameters. In some implementations, the user device 106-c may emit the sound 415 to notify the user 102-c that the wearable device 104-e was left behind. For example, the user device 106-c may play a high-pitched noise, or low-pitched noise, a frequency tone of the sound 415, a ringtone of the sound 415, or any combination thereof. In some cases, the user device 106-c may play a sound 415 that varies in volume and frequency to act as a beacon for the user 102-c to locate the relative location of the wearable device 104-c. For example, as the user 102-c nears a location of the wearable device 104-e, the tone, frequency, pattern, or the like of the sound may change (e.g., the volume may increase as the distance decreases, a periodicity of the sound may increase as the distance decreases, or the like).

In some other examples, the wearable device 104-e may emit the sound 415 to notify the user 102-c that the wearable device 104-e was left behind. For example, the wearable device 104-e may use sound haptics or a component capable of vibrating the wearable device 104-e (e.g., inside the wearable device 104-e) to emit a sound 415. In some examples, the wearable device 104-e may initiate a high or low vibration frequency, or patterns of vibrations ranging in speed, or a combination of sound haptics and vibrations. For example, the wearable device 104-e may be left on a hard surface (e.g., a table, a floor, a desk), and the user 102-c may select the trigger sounds feature 435 for the wearable device 104-e to emit vibrations or a sound 415 that create noises that the user 102-c listens for to help locate the wearable device 104-e. In some examples, the wearable device 104-e may emit vibrations or a sound 415 that vary in volume and frequency to act as a beacon for the user 102-c to locate the relative location of the wearable device 104-c. In some examples, if the user 102-c has earbuds, the earbuds may emit the sound 415 through a component of the earbud. The user device 106-c may trigger the sounds 415 regardless of whether the user 102-c selects the trigger sounds feature 435 option. For example, the user device 106-c may detect an off wear state of the wearable device 104-e and may automatically trigger one or more components to emit the sound 415 (e.g., components of the user device 106-c, the wearable device 104-e, earbuds of the user 102-c, or the like).

In some aspects, the wearable device 104-e may include an intelligent power save mode that ensures a battery life of the wearable device 104-e stays above a threshold battery level for a relatively long duration. For example, the power saving mode may ensure that the battery of the wearable device 104-e, the user device 106-c, or both, maintains power levels while using the trigger lights feature 430, the trigger sounds feature 435, or both. In some examples, the wearable device 104-e may adjust current and run (e.g., burn) through time and frequency resources efficiently (e.g., intelligently). In some cases, a power saving mode of the wearable device 104-e, the user device 106-c, or both, may refrain from using the visible light sources (e.g., the visible light sources are turned off), the sound components, or both, and may wait until the user device 106-c is within a threshold distance 410 relative to a location of the wearable device 104-e. In some instances, the user device 106-c may detect the relative location of the wearable device 104-e and may reinitiate the use of visible light sources (e.g., the visible light sources are turned on).

FIG. 5 illustrates an example of a GUI 500 that supports techniques for generating alerts based on a relative location of a wearable device in accordance with aspects of the present disclosure. The GUI 500 may implement, or be implemented by, aspects of the system 100, the system 200, the wear state diagram 300, the user diagram 400, or any combination thereof. For example, the GUI 500 may be implemented at a user device connected to a wearable device (e.g., a ring, watch, necklace, or any other wearable device), which may be examples of a user device 106 and a wearable device 104 as described with reference to FIGS. 1-4.

The GUI 500 illustrates a series of application pages, including an application page 505-a, an application page 505-b, and an application page 505-c, that may be displayed to a user via the GUI 500 (e.g., a user 102 and a GUI 275 as described with reference to FIGS. 1-4). In some examples, a user may activate a lost mode by selecting an option via the GUI, such as from the application page 505-a. For example, the application page 505-a may display a connection status message 510 and a readiness display 511 related to activity levels of the user. In some examples, the connection status message 510 may prompt the user if a wearable device is failing to connect to the user device 106 (e.g., the wearable device 104 is in an off wear state and/or the threshold distance between the wearable device 104 and the user device 106 satisfies or fails to satisfy a threshold as described with reference to FIGS. 1-4). In some examples, the user may activate a lost mode feature 515 for the wearable device 104 after receiving the connection status message 510. In some examples, the user may select a lost mode feature 515, trigger a find my ring feature 520, or both.

In some examples, the user may trigger the find my ring feature 520 to attempt to locate the wearable device 104. In some examples, an application page 505-b may display a last known location 525 of the wearable device when a signal strength between the user device 106 and the wearable device 104 fails to satisfy a threshold signal strength, as described with reference to FIG. 4. In some cases, the user device may access stored location data including a last known geographical location (e.g., a Global Positioning System (GPS) location) of the wearable device 104 when the signal strength fails to satisfy the threshold signal strength. In some examples, the application page 505-b may display a map 535 showing the geographical location of the wearable device 104 as the last known location 525 of the wearable device 104. In some cases, the map 535 may illustrate a pin 530 indicating the last known location 525 of the wearable device 104 and a user icon 531 that illustrates the position of the user 102 relative to the wearable device 104 (e.g., relative to the pin 530). In some examples, the transmittal Bluetooth features between the wearable device 104 and the user 102 may be based on a threshold distance and may not update the relative location of the wearable device 104 when the distance between the devices exceeds the threshold distance. In this example, the threshold distance of wireless communications between the user device 106 and the wearable device 104 may be indicated by the dashed line surrounding the pin 530.

In some instances, the last known location 525 may indicate to the user that the distance between the wearable device 104 and the user 102 (e.g., user device 106) has exceeded the threshold distance and may display additional statistics relating to the last known location 525. For example, the last known location 525 of the wearable device may indicate the distance away 525 (e.g., X Distance Away in kilometers (km)) from the respective user. In some cases, the last known location 525 of the wearable device 104 may indicate a time 545 (e.g., X minutes ago) when the last known location 525 was indicated (e.g., last time that the wearable device 104 and the user device 106 were communicatively coupled with one another). In some examples, the last known location 525 of the wearable device 104 may indicate a location 550 (e.g., Find My Ring at X), which may include a set of coordinates or other landmarks nearby to the wearable device 104.

In some cases, the GUI may display a second notification (e.g., another notification) when the signal strength satisfies the threshold signal strength. In some examples, the user may check the application page 505-b when returning to the last known location 525. In some cases, the wearable device 104 may be at the last known location and the user may be within range of the threshold distance. In some examples, the signal strength satisfies the threshold signal strength between the user device 106 and the wearable device 104, and the GUI may display the second notification that the wearable device 104 is within the threshold distance. For example, the application page 505-b may notify the user that they are in range of the wearable device 104 (e.g., wearable device 104 is within X meters from the user). In some implementations, the second notification that appears on the application page 505-b may provide for the user to narrow down the relative location of the wearable device 104.

In some aspects, the last known location 525 may pick up a distance and current location if the user is within the threshold distance of the wearable device 104. In some examples, an application page 505-c may display a last known location 555 of the wearable device 104 when the signal strength satisfies a threshold signal strength. In some examples, a map 565 may be displayed showing the geographical location as the last known location 555 of the wearable device 104 to the user. In some cases, the map 565 may illustrate a pin 560 indicating the current pinned location of the wearable device 104 and a user icon 532 that illustrates the position of the user relative to the wearable device 104. In some examples, the transmittal Bluetooth features between the wearable device 104 and the user device 106 may be based on a threshold distance and may update the relative location of the wearable device 104 in real time when the distance between the devices is within the threshold distance. In this example, the threshold distance between the user device 106 and the wearable device 104 may be indicated by the dashed line surrounding the pin 560.

In some instances, the last known location 555 may indicate to the user that the distance between the wearable device 104 and the user is within range of the threshold distance and may display additional statistics relating to the last known location 555. For example, the last known location 555 of the wearable device 104 may indicate the distance away 570 (e.g., Distance away: X in meters (m)) from the respective user. In some cases, the last known location 555 of the wearable device 104 may indicate a current time 575 (e.g., Current Time: X) when the last known location 555 was updated. In some examples, the last known location 555 of the wearable device 104 may indicate a current location 580 (e.g., Find My Ring at X), which may include a set of coordinates or other landmarks nearby to the wearable device 104. In some cases, the user may activate one or more features to trigger lights or sounds to locate the wearable device 104 within the threshold distance.

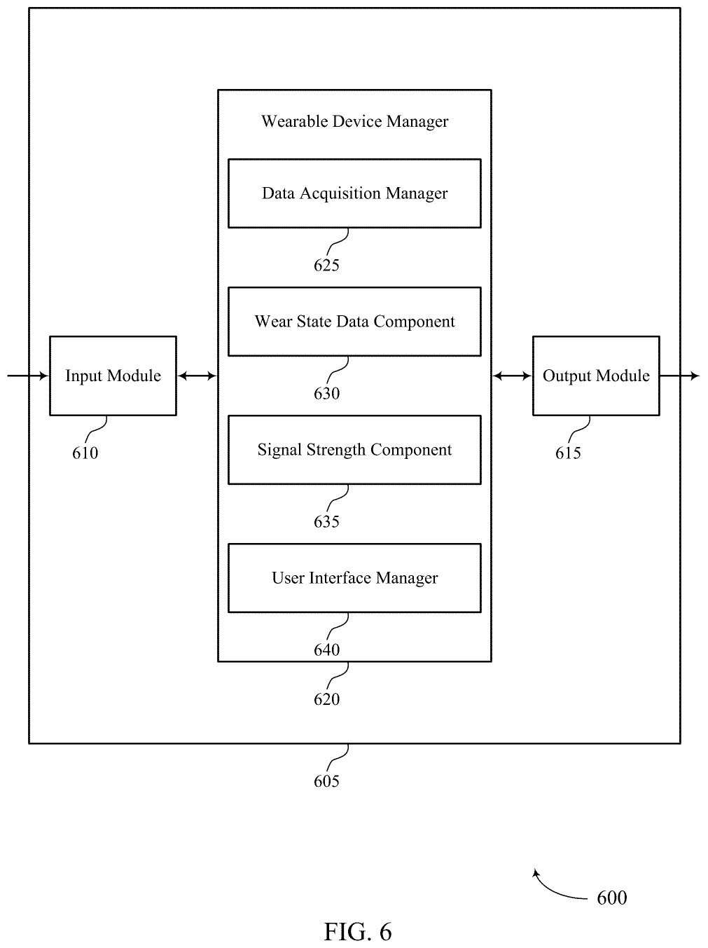

FIG. 6 illustrates a block diagram 600 of a device 605 that supports techniques for generating alerts based on a relative location of a wearable device in accordance with aspects of the present disclosure. The device 605 may include an input module 610, an output module 615, and a wearable device manager 620. The device 605 may also include a processor. Each of these components may be in communication with one another (e.g., via one or more buses).

For example, the wearable device manager 620 may include a data acquisition manager 625, a wear state data component 630, a signal strength component 635, a user interface manager 640, or any combination thereof. In some examples, the wearable device manager 620, or various components thereof, may be configured to perform various operations (e.g., receiving, monitoring, transmitting) using or otherwise in cooperation with the input module 610, the output module 615, or both. For example, the wearable device manager 620 may receive information from the input module 610, send information to the output module 615, or be integrated in combination with the input module 610, the output module 615, or both to receive information, transmit information, or perform various other operations as described herein.

The data acquisition manager 625 may be configured as or otherwise support a means for acquiring data via a wearable device associated with a user, the data comprising temperature data, acceleration data, PPG data, or any combination thereof. The wear state data component 630 may be configured as or otherwise support a means for determining that the user is not wearing the wearable device based at least in part on the data. The signal strength component 635 may be configured as or otherwise support a means for determining a signal strength associated with a wireless connection between the wearable device and a user device associated with the user based at least in part on determining that the user is not wearing the wearable device. The user interface manager 640 may be configured as or otherwise support a means for causing a GUI of the user device to display a notification associated with the wearable device based at least in part on the signal strength failing to satisfy a threshold signal strength.

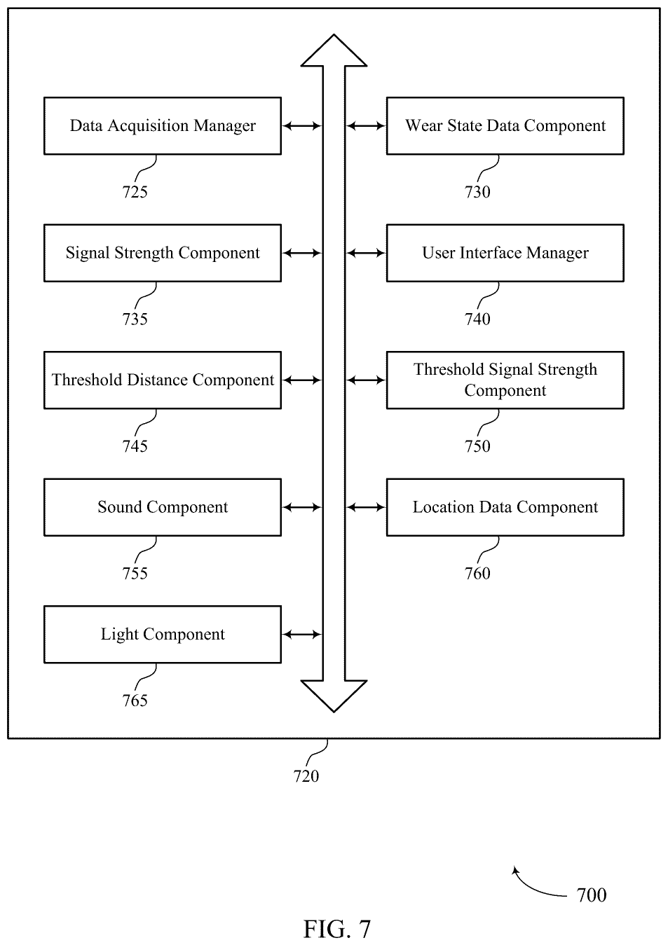

FIG. 7 illustrates a block diagram 700 of a wearable device manager 720 that supports techniques for generating alerts based on a relative location of a wearable device in accordance with aspects of the present disclosure. The wearable device manager 720 may be an example of aspects of a wearable device manager or a wearable device manager 620, or both, as described herein. The wearable device manager 720, or various components thereof, may be an example of means for performing various aspects of techniques for generating alerts based on a relative location of a wearable device as described herein. For example, the wearable device manager 720 may include a data acquisition manager 725, a wear state data component 730, a signal strength component 735, a user interface manager 740, a threshold distance component 745, a threshold signal strength component 750, a sound component 755, a location data component 760, a light component 765, or any combination thereof. Each of these components may communicate, directly or indirectly, with one another (e.g., via one or more buses).

The data acquisition manager 725 may be configured as or otherwise support a means for acquiring data via a wearable device associated with a user, the data comprising temperature data, acceleration data, PPG data, or any combination thereof. The wear state data component 730 may be configured as or otherwise support a means for determining that the user is not wearing the wearable device based at least in part on the data. The signal strength component 735 may be configured as or otherwise support a means for determining a signal strength associated with a wireless connection between the wearable device and a user device associated with the user based at least in part on determining that the user is not wearing the wearable device. The user interface manager 740 may be configured as or otherwise support a means for causing a GUI of the user device to display a notification associated with the wearable device based at least in part on the signal strength failing to satisfy a threshold signal strength.

In some examples, the threshold distance component 745 may be configured as or otherwise support a means for receiving, via the user device, a user input indicating a threshold distance between the user device and the wearable device. In some examples, the threshold signal strength component 750 may be configured as or otherwise support a means for determining the threshold signal strength based at least in part on the threshold distance.

In some examples, the user interface manager 740 may be configured as or otherwise support a means for causing the GUI of the user device to display a second notification based at least in part on the signal strength satisfying the threshold signal strength.

In some examples, to support determining that the user is not wearing the wearable device, the wear state data component 730 may be configured as or otherwise support a means for determining the temperature data, motion data, the PPG data, ECG data, bioimpedance data, or any combination thereof, satisfy one or more threshold values, the one or more threshold values corresponding to the user not wearing the wearable device.

In some examples, to support determining that the user is not wearing the wearable device, the wear state data component 730 may be configured as or otherwise support a means for determining that a change in the acceleration data satisfies a threshold acceleration value.

In some examples, the sound component 755 may be configured as or otherwise support a means for causing one or more components of the user device to emit a sound based at least in part on the signal strength failing to satisfy the threshold signal strength.

In some examples, the sound component 755 may be configured as or otherwise support a means for selectively adjusting one or more parameters associated with the sound based at least in part on a distance between the user device and the wearable device, a power level of the wearable device, or both, wherein the distance is associated with the signal strength, and wherein the one or more parameters comprise a volume of the sound, a frequency of the sound, a pattern of the sound, or any combination thereof.

In some examples, the location data component 760 may be configured as or otherwise support a means for storing location data associated with a geographical location of the wearable device, the user device, or both, based at least in part on the signal strength failing to satisfy the threshold signal strength. In some examples, the user interface manager 740 may be configured as or otherwise support a means for causing the GUI to display an indication of the geographical location.

In some examples, the light component 765 may be configured as or otherwise support a means for causing one or more components of the wearable device to emit a visible light based at least in part on the signal strength failing to satisfy the threshold signal strength.

In some examples, the light component 765 may be configured as or otherwise support a means for selectively adjusting one or more parameters associated with the visible light based at least in part on a distance between the user device and the wearable device, a power level of the wearable device, or both, wherein the distance is associated with the signal strength, and wherein the one or more parameters comprise an intensity of the visible light, a wavelength associated with the visible light, a blinking pattern associated with the visible light, a periodicity of the blinking pattern, or any combination thereof.

In some examples, to support causing the one or more components of the wearable device to emit the visible light, the light component 765 may be configured as or otherwise support a means for determining a distance between the user device and the wearable device satisfies a threshold distance based at least in part on the signal strength.

In some examples, the wireless connection comprises a Bluetooth connection.

In some examples, the wearable device comprises a wearable ring device.

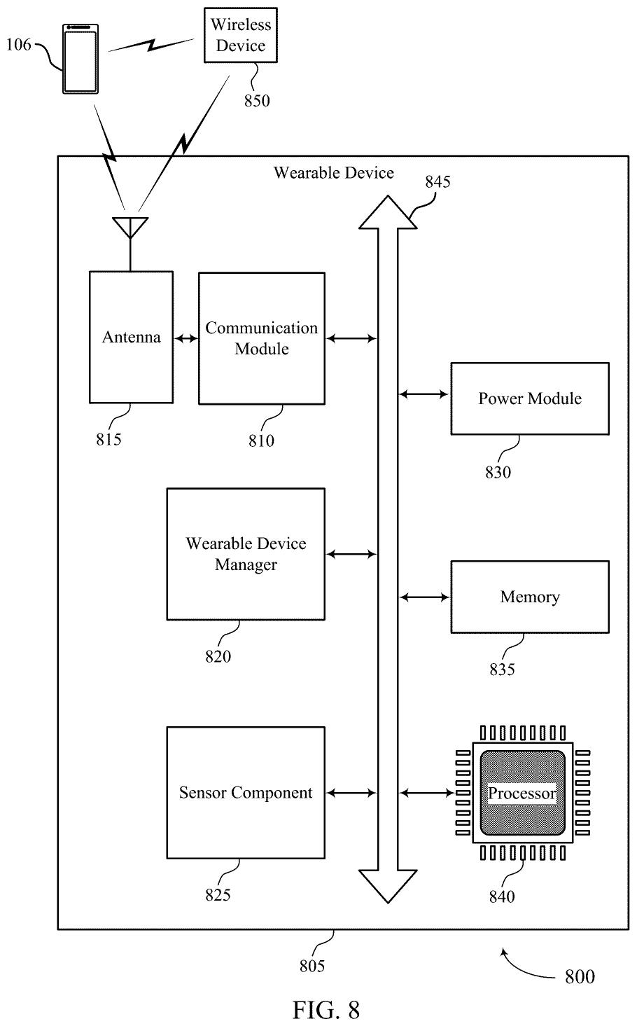

FIG. 8 illustrates a diagram of a system 800 including a device 805 that supports techniques for generating alerts based on a relative location of a wearable device in accordance with aspects of the present disclosure. The device 805 may be an example of or include the components of a device 605 as described herein. The device 805 may include an example of a wearable device 104, as described previously herein. The device 805 may include components for bi-directional communications including components for transmitting and receiving communications with a user device 106 and a server 110, such as a wearable device manager 820, a communication module 810, an antenna 815, a sensor component 825, a power module 830, a memory 835, a processor 840, and a wireless device 850. These components may be in electronic communication or otherwise coupled (e.g., operatively, communicatively, functionally, electronically, electrically) via one or more buses (e.g., a bus 845).

For example, the wearable device manager 820 may be configured as or otherwise support a means for acquiring data via a wearable device associated with a user, the data comprising temperature data, acceleration data, PPG data, or any combination thereof. The wearable device manager 820 may be configured as or otherwise support a means for determining that the user is not wearing the wearable device based at least in part on the data. The wearable device manager 820 may be configured as or otherwise support a means for determining a signal strength associated with a wireless connection between the wearable device and a user device associated with the user based at least in part on determining that the user is not wearing the wearable device. The wearable device manager 820 may be configured as or otherwise support a means for causing a GUI of the user device to display a notification associated with the wearable device based at least in part on the signal strength failing to satisfy a threshold signal strength.

By including or configuring the wearable device manager 820 in accordance with examples as described herein, the device 805 may support techniques for generating alerts based on a relative location of the wearable device. In some examples, the techniques may prevent loss of the wearable device and false notifications that the wearable device is lost. In some aspects, the techniques may improve user experience and prevent users from spending money on additional wearable devices to replace a lost wearable device.

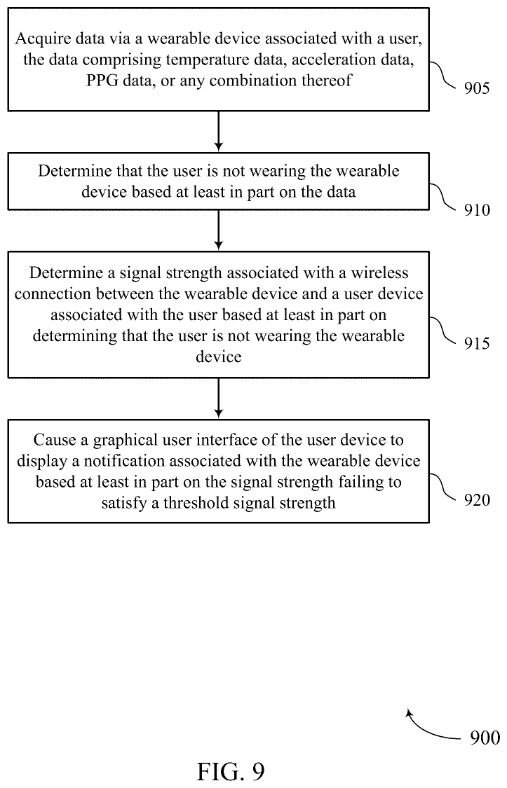

FIG. 9 illustrates a flowchart showing a method 900 that supports techniques for generating alerts based on a relative location of a wearable device in accordance with aspects of the present disclosure. The operations of the method 900 may be implemented by a wearable device or its components as described herein. For example, the operations of the method 900 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 905, the method may include acquiring data via a wearable device associated with a user, the data comprising temperature data, acceleration data, PPG data, or any combination thereof. The operations of 905 may be performed in accordance with examples as disclosed herein. In some examples, aspects of the operations of 905 may be performed by a data acquisition manager 725 as described with reference to FIG. 7.

At 910, the method may include determining that the user is not wearing the wearable device based at least in part on the data. The operations of 910 may be performed in accordance with examples as disclosed herein. In some examples, aspects of the operations of 910 may be performed by a wear state data component 730 as described with reference to FIG. 7.

At 915, the method may include determining a signal strength associated with a wireless connection between the wearable device and a user device associated with the user based at least in part on determining that the user is not wearing the wearable device. The operations of 915 may be performed in accordance with examples as disclosed herein. In some examples, aspects of the operations of 915 may be performed by a signal strength component 735 as described with reference to FIG. 7.

At 920, the method may include causing a GUI of the user device to display a notification associated with the wearable device based at least in part on the signal strength failing to satisfy a threshold signal strength. The operations of 920 may be performed in accordance with examples as disclosed herein. In some examples, aspects of the operations of 920 may be performed by a user interface manager 740 as described with reference to FIG. 7.

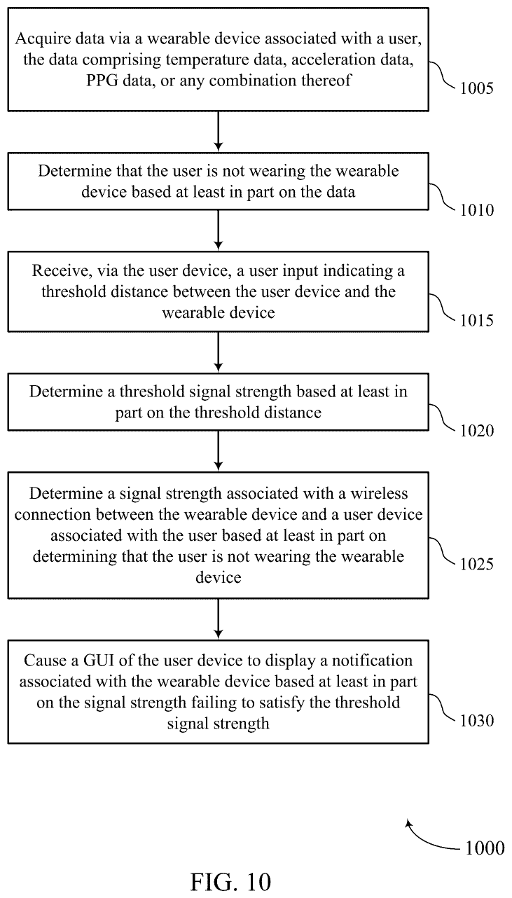

FIG. 10 illustrates a flowchart showing a method 1000 that supports techniques for generating alerts based on a relative location of a wearable device in accordance with aspects of the present disclosure. The operations of the method 1000 may be implemented by a wearable device or its components as described herein. For example, the operations of the method 1000 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 1005, the method may include acquiring data via a wearable device associated with a user, the data comprising temperature data, acceleration data, PPG data, or any combination thereof. The operations of 1005 may be performed in accordance with examples as disclosed herein. In some examples, aspects of the operations of 1005 may be performed by a data acquisition manager 725 as described with reference to FIG. 7.

At 1010, the method may include determining that the user is not wearing the wearable device based at least in part on the data. The operations of 1010 may be performed in accordance with examples as disclosed herein. In some examples, aspects of the operations of 1010 may be performed by a wear state data component 730 as described with reference to FIG. 7.

At 1015, the method may include receiving, via the user device, a user input indicating a threshold distance between the user device and the wearable device. The operations of 1015 may be performed in accordance with examples as disclosed herein. In some examples, aspects of the operations of 1015 may be performed by a threshold distance component 745 as described with reference to FIG. 7.

At 1020, the method may include determining a threshold signal strength based at least in part on the threshold distance. The operations of 1020 may be performed in accordance with examples as disclosed herein. In some examples, aspects of the operations of 1020 may be performed by a threshold signal strength component 750 as described with reference to FIG. 7.

At 1025, the method may include determining a signal strength associated with a wireless connection between the wearable device and a user device associated with the user based at least in part on determining that the user is not wearing the wearable device. The operations of 1025 may be performed in accordance with examples as disclosed herein. In some examples, aspects of the operations of 1025 may be performed by a signal strength component 735 as described with reference to FIG. 7.

At 1030, the method may include causing a GUI of the user device to display a notification associated with the wearable device based at least in part on the signal strength failing to satisfy the threshold signal strength. The operations of 1030 may be performed in accordance with examples as disclosed herein. In some examples, aspects of the operations of 1030 may be performed by a user interface manager 740 as described with reference to FIG. 7.

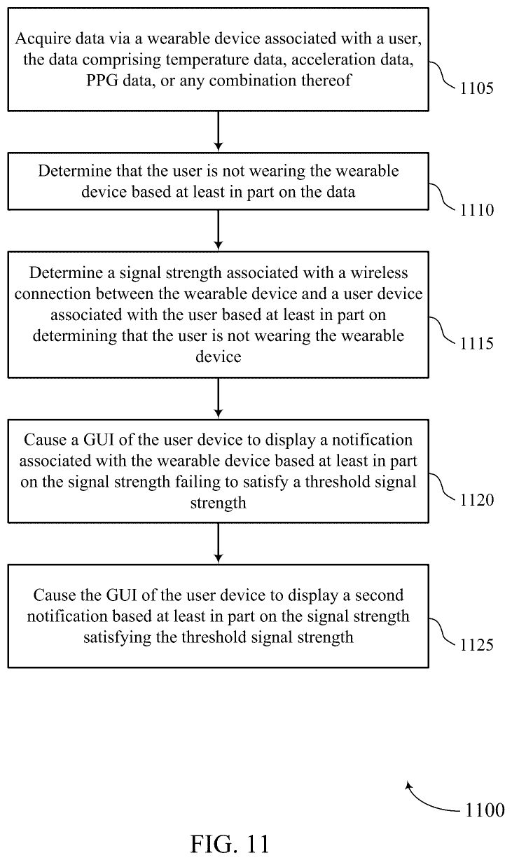

FIG. 11 illustrates a flowchart showing a method 1100 that supports techniques for generating alerts based on a relative location of a wearable device in accordance with aspects of the present disclosure. The operations of the method 1100 may be implemented by a wearable device or its components as described herein. For example, the operations of the method 1100 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 1105, the method may include acquiring data via a wearable device associated with a user, the data comprising temperature data, acceleration data, PPG data, or any combination thereof. The operations of 1105 may be performed in accordance with examples as disclosed herein. In some examples, aspects of the operations of 1105 may be performed by a data acquisition manager 725 as described with reference to FIG. 7.

At 1110, the method may include determining that the user is not wearing the wearable device based at least in part on the data. The operations of 1110 may be performed in accordance with examples as disclosed herein. In some examples, aspects of the operations of 1110 may be performed by a wear state data component 730 as described with reference to FIG. 7.

At 1115, the method may include determining a signal strength associated with a wireless connection between the wearable device and a user device associated with the user based at least in part on determining that the user is not wearing the wearable device. The operations of 1115 may be performed in accordance with examples as disclosed herein. In some examples, aspects of the operations of 1115 may be performed by a signal strength component 735 as described with reference to FIG. 7.

At 1120, the method may include causing a GUI of the user device to display a notification associated with the wearable device based at least in part on the signal strength failing to satisfy a threshold signal strength. The operations of 1120 may be performed in accordance with examples as disclosed herein. In some examples, aspects of the operations of 1120 may be performed by a user interface manager 740 as described with reference to FIG. 7.

At 1125, the method may include causing the GUI of the user device to display a second notification based at least in part on the signal strength satisfying the threshold signal strength. The operations of 1125 may be performed in accordance with examples as disclosed herein. In some examples, aspects of the operations of 1125 may be performed by a user interface manager 740 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method is described. The method may include acquiring data via a wearable device associated with a user, the data comprising temperature data, acceleration data, PPG data, or any combination thereof, determining that the user is not wearing the wearable device based at least in part on the data, determining a signal strength associated with a wireless connection between the wearable device and a user device associated with the user based at least in part on determining that the user is not wearing the wearable device, and causing a GUI of the user device to display a notification associated with the wearable device based at least in part on the signal strength failing to satisfy a threshold signal strength.

An apparatus is described. The apparatus may include a processor, memory coupled with the processor, and instructions stored in the memory. The instructions may be executable by the processor to cause the apparatus to acquire data via a wearable device associated with a user, the data comprising temperature data, acceleration data, PPG data, or any combination thereof, determine that the user is not wearing the wearable device based at least in part on the data, determine a signal strength associated with a wireless connection between the wearable device and a user device associated with the user based at least in part on determining that the user is not wearing the wearable device, and cause a GUI of the user device to display a notification associated with the wearable device based at least in part on the signal strength failing to satisfy a threshold signal strength.

Another apparatus is described. The apparatus may include means for acquiring data via a wearable device associated with a user, the data comprising temperature data, acceleration data, PPG data, or any combination thereof, means for determining that the user is not wearing the wearable device based at least in part on the data, means for determining a signal strength associated with a wireless connection between the wearable device and a user device associated with the user based at least in part on determining that the user is not wearing the wearable device, and means for causing a GUI of the user device to display a notification associated with the wearable device based at least in part on the signal strength failing to satisfy a threshold signal strength.

A non-transitory computer-readable medium storing code is described. The code may include instructions executable by a processor to acquire data via a wearable device associated with a user, the data comprising temperature data, acceleration data, PPG data, or any combination thereof, determine that the user is not wearing the wearable device based at least in part on the data, determine a signal strength associated with a wireless connection between the wearable device and a user device associated with the user based at least in part on determining that the user is not wearing the wearable device, and cause a GUI of the user device to display a notification associated with the wearable device based at least in part on the signal strength failing to satisfy a threshold signal strength.

Some examples of the method, apparatuses, and non-transitory computer-readable medium described herein may further include operations, features, means, or instructions for receiving, via the user device, a user input indicating a threshold distance between the user device and the wearable device and determining the threshold signal strength based at least in part on the threshold distance.

Some examples of the method, apparatuses, and non-transitory computer-readable medium described herein may further include operations, features, means, or instructions for causing the GUI of the user device to display a second notification based at least in part on the signal strength satisfying the threshold signal strength.

In some examples of the method, apparatuses, and non-transitory computer-readable medium described herein, determining that the user may be not wearing the wearable device may include operations, features, means, or instructions for determining the temperature data, motion data, the PPG data, ECG data, bioimpedance data, or any combination thereof, satisfy one or more threshold values, the one or more threshold values corresponding to the user not wearing the wearable device.

In some examples of the method, apparatuses, and non-transitory computer-readable medium described herein, determining that the user may be not wearing the wearable device may include operations, features, means, or instructions for determining that a change in the acceleration data satisfies a threshold acceleration value.

Some examples of the method, apparatuses, and non-transitory computer-readable medium described herein may further include operations, features, means, or instructions for causing one or more components of the user device to emit a sound based at least in part on the signal strength failing to satisfy the threshold signal strength.

In some examples of the method, apparatuses, and non-transitory computer-readable medium described herein, selectively adjusting one or more parameters associated with the sound based at least in part on a distance between the user device and the wearable device, a power level of the wearable device, or both, wherein the distance may be associated with the signal strength, and wherein the one or more parameters comprise a volume of the sound, a frequency of the sound, a pattern of the sound, or any combination thereof.

Some examples of the method, apparatuses, and non-transitory computer-readable medium described herein may further include operations, features, means, or instructions for storing location data associated with a geographical location of the wearable device, the user device, or both, based at least in part on the signal strength failing to satisfy the threshold signal strength and causing the GUI to display an indication of the geographical location.

Some examples of the method, apparatuses, and non-transitory computer-readable medium described herein may further include operations, features, means, or instructions for causing one or more components of the wearable device to emit a visible light based at least in part on the signal strength failing to satisfy the threshold signal strength.

In some examples of the method, apparatuses, and non-transitory computer-readable medium described herein, selectively adjusting one or more parameters associated with the visible light based at least in part on a distance between the user device and the wearable device, a power level of the wearable device, or both, wherein the distance may be associated with the signal strength, and wherein the one or more parameters comprise an intensity of the visible light, a wavelength associated with the visible light, a blinking pattern associated with the visible light, a periodicity of the blinking pattern, or any combination thereof.

In some examples of the method, apparatuses, and non-transitory computer-readable medium described herein, causing the one or more components of the wearable device to emit the visible light may include operations, features, means, or instructions for determining a distance between the user device and the wearable device satisfies a threshold distance based at least in part on the signal strength.

In some examples of the method, apparatuses, and non-transitory computer-readable medium described herein, the wireless connection comprises a Bluetooth connection.

In some examples of the method, apparatuses, and non-transitory computer-readable medium described herein, the wearable device comprises a wearable ring device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method comprising:
acquiring data via a wearable device associated with a user, the data comprising temperature data, acceleration data, photoplethysmogram (PPG) data, or any combination thereof;
determining that the user is not wearing the wearable device based at least in part on the data;
determining a signal strength associated with a wireless connection between the wearable device and a user device associated with the user based at least in part on determining that the user is not wearing the wearable device; and
causing a graphical user interface of the user device to display a notification associated with the wearable device based at least in part on the signal strength failing to satisfy a threshold signal strength.

2. The method of claim 1, further comprising:
receiving, via the user device, a user input indicating a threshold distance between the user device and the wearable device; and
determining the threshold signal strength based at least in part on the threshold distance.

3. The method of claim 1, further comprising:
causing the graphical user interface of the user device to display a second notification based at least in part on the signal strength satisfying the threshold signal strength.

4. The method of claim 1, wherein determining that the user is not wearing the wearable device further comprises:
determining the temperature data, motion data, the PPG data, electrocardiogram data, bioimpedance data, or any combination thereof, satisfy one or more threshold values, the one or more threshold values corresponding to the user not wearing the wearable device.

5. The method of claim 1, wherein determining that the user is not wearing the wearable device further comprises:
determining that a change in the acceleration data satisfies a threshold acceleration value.

6. The method of claim 1, further comprising:
causing one or more components of the user device to emit a sound based at least in part on the signal strength failing to satisfy the threshold signal strength.

7. The method of claim 6, further comprising:
selectively adjusting one or more parameters associated with the sound based at least in part on a distance between the user device and the wearable device, a power level of the wearable device, or both, wherein the distance is associated with the signal strength, and wherein the one or more parameters comprise a volume of the sound, a frequency of the sound, a pattern of the sound, or any combination thereof.

8. The method of claim 1, further comprising:
storing location data associated with a geographical location of the wearable device, the user device, or both, based at least in part on the signal strength failing to satisfy the threshold signal strength; and
causing the graphical user interface to display an indication of the geographical location.

9. The method of claim 1, further comprising:
causing one or more components of the wearable device to emit a visible light based at least in part on the signal strength failing to satisfy the threshold signal strength.

10. The method of claim 9, further comprising:
selectively adjusting one or more parameters associated with the visible light based at least in part on a distance between the user device and the wearable device, a power level of the wearable device, or both, wherein the distance is associated with the signal strength, and wherein the one or more parameters comprise an intensity of the visible light, a wavelength associated with the visible light, a blinking pattern associated with the visible light, a periodicity of the blinking pattern, or any combination thereof.

11. The method of claim 9, wherein causing the one or more components of the wearable device to emit the visible light comprises:
determining a distance between the user device and the wearable device satisfies a threshold distance based at least in part on the signal strength.

12. The method of claim 1, wherein the wireless connection comprises a Bluetooth connection.

13. The method of claim 1, wherein the wearable device comprises a wearable ring device.

14. An apparatus, comprising:
a processor;
memory coupled with the processor; and
instructions stored in the memory and executable by the processor to cause the apparatus to:
acquire data via a wearable device associated with a user, the data comprising temperature data, acceleration data, photoplethysmogram (PPG) data, or any combination thereof;
determine that the user is not wearing the wearable device based at least in part on the data;
determine a signal strength associated with a wireless connection between the wearable device and a user device associated with the user based at least in part on determining that the user is not wearing the wearable device; and
cause a graphical user interface of the user device to display a notification associated with the wearable device based at least in part on the signal strength failing to satisfy a threshold signal strength.

15. The apparatus of claim 14, wherein the instructions are further executable by the processor to cause the apparatus to:
receive, via the user device, a user input indicating a threshold distance between the user device and the wearable device; and
determine the threshold signal strength based at least in part on the threshold distance.

16. The apparatus of claim 14, wherein the instructions are further executable by the processor to cause the apparatus to:
cause the graphical user interface of the user device to display a second notification based at least in part on the signal strength satisfying the threshold signal strength.

17. The apparatus of claim 14, wherein the instructions to determine that the user is not wearing the wearable device are further executable by the processor to cause the apparatus to:
determine the temperature data, motion data, the PPG data, electrocardiogram data, bioimpedance data, or any combination thereof, satisfy one or more threshold values, the one or more threshold values corresponding to the user not wearing the wearable device.

18. The apparatus of claim 14, wherein the instructions to determine that the user is not wearing the wearable device are further executable by the processor to cause the apparatus to:
determine that a change in the acceleration data satisfies a threshold acceleration value.

19. The apparatus of claim 14, wherein the instructions are further executable by the processor to cause the apparatus to:
cause one or more components of the user device to emit a sound based at least in part on the signal strength failing to satisfy the threshold signal strength.

20. A non-transitory computer-readable medium storing code, the code comprising instructions executable by a processor to:
acquire data via a wearable device associated with a user, the data comprising temperature data, acceleration data, photoplethysmogram (PPG) data, or any combination thereof;
determine that the user is not wearing the wearable device based at least in part on the data;
determine a signal strength associated with a wireless connection between the wearable device and a user device associated with the user based at least in part on determining that the user is not wearing the wearable device; and
cause a graphical user interface of the user device to display a notification associated with the wearable device based at least in part on the signal strength failing to satisfy a threshold signal strength.
